(19) United States
(12) Patent Application Publication
(10) Pub. No.: US 2025/0232554 A1
(43) Pub. Date: Jul. 17, 2025

(54) METHOD FOR DISPLAYING REGION OF INTEREST BY COMPARING MEDICAL DATA

(71) Applicant: IRM Inc., Seoul (KR)

(72) Inventor: Seung Wook CHOI, Seongnam-si (KR)

(73) Assignee: IRM Inc., Seoul (KR)

(21) Appl. No.: 19/006,058

(22) Filed: Dec. 30, 2024

(30) Foreign Application Priority Data
Dec. 28, 2023 (KR) 10-2023-0193847
Feb. 28, 2024 (KR) 10-2024-0028447

(51) Int. Cl. G06V 10/25; A61B 6/00; A61B 6/46; G16H 15/00

(52) U.S. Cl. CPC G06V 10/25; G16H 15/00; A61B 6/463; A61B 6/5205; G06V 2201/03

(57) ABSTRACT

Provided is a method of displaying a region of interest (ROI) of target data based on a comparison of medical data, the method comprising: producing a comparison value based on a difference between comparison data and the target data, identifying an ROI in an object of interest included in the target data based on the comparison value and the target data and displaying the identified ROI in a different display method from remaining regions excluding the ROI.

### CROSS-REFERENCE TO RELATED APPLICATION

The present application is based upon and claims the benefit of priority to Korean Patent Application No. 10-2023-0193847, filed on Dec. 28, 2023, and Korean Patent Application No. 10-2024-0028447, filed on Feb. 28, 2024. The disclosures of the above-listed applications are hereby incorporated by reference herein in their entirety.

### TECHNICAL FIELD

The technical idea of the present disclosure relates to a method of displaying a region of interest, and more specifically, to a method of displaying a region of interest based on comparison of medical data.

### BACKGROUND TECHNOLOGY

In conventional film-based radiology, setting of a region-of-interest (ROI) is limited to computed tomography (CT) consoles or image-reconstruction workstations. With the use of a picture archiving and communicating system (PACS) in digital radiology, there is an increasing demand to set an ROI on a PACS viewer for various purposes. When setting an ROI, statistical values of the ROI, including an area value, a mean value, a standard deviation value, a maximum value, and a minimum value, are displayed on the screen.

Radiologists read the values on the PACS screen and type the values into a required radiology report. Sometimes, especially for research purposes, a radiologist needs to set a very large number of ROIs and manually enter the relevant values directly into a spreadsheet. However, the manual processing is a repetitive and tedious task, and typos may occur during the process.

### SUBJECT MATTER OF THE INVENTION

### Problem to be Solved

The present invention is directed to providing a method of displaying a region of interest required by a user based on a comparison of medical data.

### Means for Solving the Problem

According to an aspect of the present invention, there is provided a method of displaying an ROI (ROI) based on a comparison of medical data, which includes: producing a comparison value based on a difference between comparison data and target data; comparing the comparison value with a reference value that varies for each object, identifying an ROI for the object when the comparison value is greater than or equal to the reference value; and displaying the ROI of the object of interest, which is identified based on the target data, in a different display method from remaining regions excluding the ROI.

The producing of the comparison value may include: when the comparison data and the target data are medical reports, extracting object information included in the report and a numerical value corresponding to the object information; and producing a difference between a numerical value included in the comparison data and a numerical value included in the target data as the comparison value.

The producing of the comparison value may include: when the comparison data and the target data are medical images, identifying an object from the medical images, measuring a size of the object using a measuring tool for each type of medical image; and producing the comparison value based on the measured size of the object.

The displaying may include: comparing a plurality of reference values for the object with a comparison value; when the comparison value is greater than or equal to a first reference value, setting a region of the object of interest that is greater than or equal to the first reference value and less than a second reference value as a first ROI; when the comparison value is greater than or equal to the second reference value, setting a region of the object of interest that is greater than or equal to the second reference value as a second ROI; and displaying the first ROI and the second ROI in different methods.

The comparing may include setting the reference value based on a normal value for the object and a value of the comparison data when the comparison data is a previous medical image.

### Effects of the Invention

The embodiments of the present disclosure can automatically set areas of interest in the target data by comparing target data with comparison data. Consequently, the analysis processor can use the automatically set areas of interest for subsequent analysis of patient conditions, and specialists can focus on reviewing medical data centered around the areas set as of interest without examining all medical data, significantly reducing analysis and review time. The effects obtainable from the exemplary embodiments of this disclosure are not limited to those mentioned above. Other effects not mentioned can be clearly derived and understood by those skilled in the art from the description below. That is, unintended effects that may arise from the implementation of the exemplary embodiments can also be derived by those skilled in the relevant technical field from the exemplary embodiments.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating components of an electronic device for automatically setting a region of interest (ROI) according to an embodiment of the present disclosure;

FIG. 2 is a flowchart showing a method of automatically setting an ROI according to an embodiment;

FIG. 3 is a diagram illustrating an embodiment in which an object of interest is extracted based on a comparison between images according to an embodiment;

FIGS. 4A to FIG. 4C are diagrams illustrating an embodiment in which an ROI is displayed based on a comparison between a comparison value and a reference value according to an embodiment; and

FIG. 5 is a diagram illustrating an embodiment in which an object of interest is extracted based on a comparison between medical reports according to an embodiment.

### DETAILED DESCRIPTION OF EXEMPLARY EMBODIMENTS

Hereinafter, embodiments of the present specification will be described in detail with reference to the accompanying drawings. While embodiments according to the present disclosure are susceptible to various modifications and alternative forms, specific embodiments thereof are shown by way of embodiment in the drawings and will herein be described in detail. It should be understood, however, that there is no intent to limit the embodiments according to the concept of the present disclosure to the particular forms disclosed, but on the contrary, the embodiments according to the concept of the present disclosure are to cover all modifications, equivalents, and alternatives falling within the spirit and scope of the invention. Like numbers refer to like elements throughout the description of the drawings.

It will be further understood that the terms “comprise,” “comprising,” “include” and/or “including” used herein specify the presence of stated features, integers, steps, operations, elements, and/or components, but do not preclude the presence or addition of one or more other features, integers, steps, operations, elements, components, and/or groups thereof.

In various embodiments of the present disclosure, the expression “or” and the like include any and all combinations of the items listed together in the corresponding phrase. For example, “A or B” may include A, may include B, or may include both A and B.

The expressions “first,” “second,” etc., used in various embodiments of the present disclosure may modify various components of various embodiments, but do not limit the components. For example, the terms do not limit the sequence and/or importance of the components, and may be used to distinguish one component from another.

It will be understood that when an element is referred to as being “connected” or “coupled” to another element, the element can be directly connected or coupled to the other element or intervening elements may be present.

In the embodiments of the present disclosure, terms such as “module,” “unit,” “part,” etc., are used to refer to components that perform at least one function or operation, and these components may be implemented in hardware or software, or as a combination of hardware and software. In addition, a plurality of “modules,” “units,” “parts,” etc., may be integrated into at least one module or chip and implemented as at least one processor, except in cases in which each needs to be implemented as specific individual hardware.

It will be further understood that terms, such as those defined in commonly used dictionaries, should be interpreted as having meanings that are consistent with their meanings in the context of the relevant art and will not be interpreted in an idealized or overly formal sense unless expressly so defined herein.

Hereinafter, an embodiment of the present disclosure will be described in detail with reference to the attached drawings.

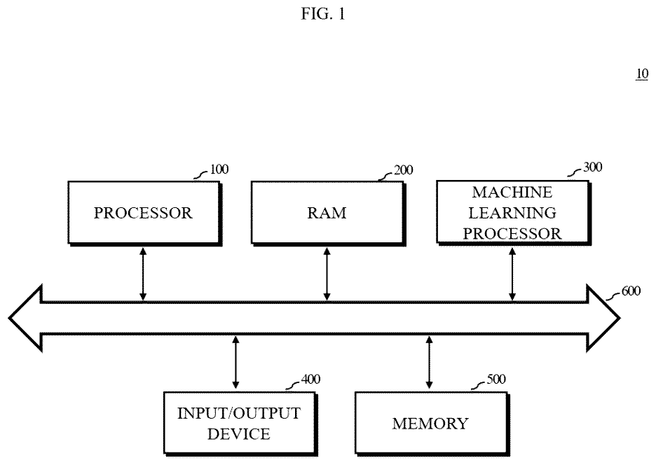

FIG. 1 is a block diagram illustrating components of an electronic device that automatically sets a region of interest according to an embodiment of the present disclosure.

Referring to FIG. 1, an electronic device 10 may compare target data with comparison data and determine whether to display at least a portion of an object of interest as a region of interest based on a comparison value produced as a result of the comparison. For example, the electronic device 10 may be applied to a computing device, a personal computer, a smart TV, a smart phone, a mobile device, a video display device, a measuring device, an Internet of things (IoT) device, etc., and may also be mounted on one of various types of electronic devices.

The electronic device 10 may include at least one IP block and a machine learning processor 300. The electronic device 10 may include various types of IP blocks, and for example, as illustrated in FIG. 1, the electronic device 10 may include IP blocks such as a processor 100, a random access memory (RAM) 200, an input/output device 400, and a memory 500. In addition, the electronic device 10 may further include other general-purpose components such as a multi-format codec (MFC), a video module (e.g., a camera interface, a joint photographic experts group (JPEG) processor, a video processor, or a mixer), a 3D graphics core, an audio system, a display driver, a graphics processing unit (GPU), a digital signal processor (DSP), and the like.

Components of the electronic device 10, such as the processor 100, the RAM 200, the machine learning processor 300, the input/output device 400, and the memory 500, may transmit and receive data through a system bus 600. For example, as a standard bus specification, the advanced microcontroller bus architecture (AMBA) protocol of advanced RISC machine (ARM) may be applied to the system bus 600. However, it is not limited thereto, and various types of protocols may be applied.

In the embodiment, the components of the electronic device 10, the processor 100, the RAM 200, the machine learning processor 300, the input/output device 400, and the memory 500 may be implemented as a single semiconductor chip, and for example, the electronic device 10 may be implemented as a system on chip (SoC). However, it is not limited thereto, and the electronic device 10 may be implemented as a plurality of semiconductor chips. In an embodiment, the electronic device 10 may be implemented as an application processor mounted on a mobile device.

The processor 100 may control the overall operation of the electronic device 10, and as an example, the processor 100 may include at least one of a central processing unit (CPU) and a GPU. The processor 100 may include one core (single core) or multiple cores (multi-core). The processor 100 may process or execute programs and/or data stored in the RAM 200 and the memory 500. For example, the processor 100 may control various functions of the electronic device 10 by executing programs stored in the memory 500.

The RAM 200 may temporarily store programs, data, or instructions. For example, programs and/or data stored in the memory 500 may be temporarily loaded into the RAM 200 according to the control or booting code of the processor 100. The RAM 200 may be implemented using a memory such as a dynamic RAM (DRAM) or a static RAM (SRAM).

The input/output device 400 may receive input data from a user or from the outside, and output a data processing result of the electronic device 10. The input/output device 400 may be implemented using at least one of a touch screen panel, a keyboard, and various types of sensors. In an embodiment, the input/output device 400 may collect information around the electronic device 10. For example, the input/output device 400 may include at least one of various types of sensing devices such as an imaging device, an image sensor, a light detection and ranging (LiDAR) sensor, an ultrasonic sensor, and an infrared sensor, or may receive a sensing signal from the device. In an embodiment, the input/output device 400 may sense or receive an image signal from the outside of the electronic device 10, and may convert the sensed or received image signal into image data, i.e., an image frame. The input/output device 400 may store the image frame in the memory 500 or provide the image frame to the machine learning processor 300.

The memory 500 is a storage location for storing data, and may store, for example, an operating system (OS), various programs, and various data. The memory 500 may be a DRAM, but is not limited thereto. The memory 500 may include at least one of a volatile memory and a non-volatile memory. The non-volatile memory may include a read only memory (ROM), a programmable ROM (PROM), an erasable programmable ROM (EPROM), an electrically erasable programmable ROM (EEPROM), a flash memory, a phase-change RAM (PRAM), a magnetic RAM (MRAM), a resistive RAM (RRAM), a ferroelectric RAM (FRAM), etc. The volatile memory may include a DRAM, an SRAM, a synchronous DRAM (SDRAM), etc. In addition, in an embodiment, the memory 150 may be implemented as a storage device such as a hard disk drive (HDD), a solid-state drive (SSD), a compact flash (CF), a secure digital (SD), a micro secure digital (Micro-SD), a Mini-SD, an extreme digital (xD), or a Memory Stick.

The machine learning processor 300 may train at least one of various types of machine learning models based on previously acquired training data, and may perform calculations based on the trained model. For example, the machine learning processor 300 may perform calculations based on received input data to generate inference values as calculation results, or retrain the machine learning model.

The types of machine learning models trained and inferred by the machine learning processor 300 may include supervised learning models, unsupervised learning models, and reinforcement learning models, and various types of models may be ensembled.

In addition, the machine learning processor 300 may generate a neural network model, train or learn a neural network, perform calculations based on received input data and generate information signals based on calculation results, or retrain a neural network. The neural network may include various types of neural network models such as a convolution neural network (CNN), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) network, a classification network, etc., but is not limited thereto.

The electronic device 10 according to the embodiment of the present disclosure may perform preprocessing on input data by the processor 100. Preprocessing of the input data may be a process for effectively processing the collected data according to the purpose. For example, the processor 100 may perform data cleaning, data transformation, data filtering, data integration, and data reduction on medical images.

The processor 100 of the electronic device 10 according to the embodiment of the present disclosure may produce a comparison value based on a difference between comparison data and target data, and the machine learning processor 300 may identify an object of interest and a region of interest based on the produced comparison value. The processor 100 may display the target data in a predetermined specific display method based on the identified object of interest and the identified region of interest.

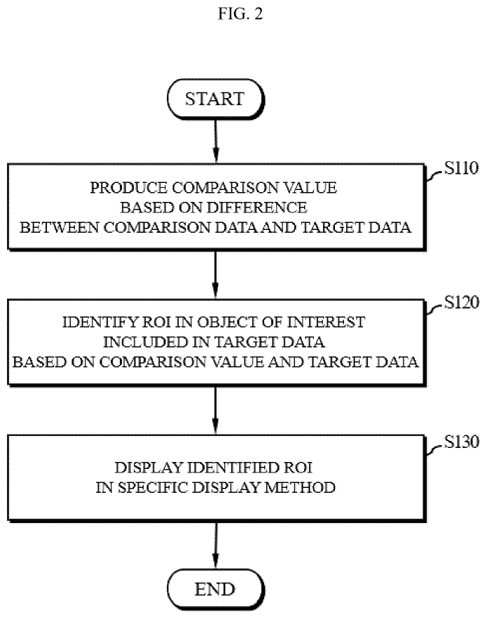

FIG. 2 is a flowchart showing a method of automatically setting an ROI according to an embodiment.

Referring to FIG. 2, the electronic device 10 of the present disclosure may produce a difference between comparison data and target data, and may set a specific portion of the target data as a region of interest (ROI) based on the produced comparison value and display the ROI in a specific display method.

Since the target data is displayed as an ROI, an analysis device or a specialist may analyze only the ROI. The comparison data may be data associated with the target data, and for example, may be previous data extracted from the same patient as the target data, or heterogeneous type data for a lesion.

In operation S110, the electronic device 10 may produce a comparison value based on a difference between the comparison data and the target data. For example, the comparison data and the target data may be medical images, and the electronic device 10 may identify at least one object included in the medical images. The object may be a part of a body tissue included in the medical image. The electronic device 10 may measure a numerical value of the at least one object using a measuring tool. The electronic device 10 may produce a difference between a numerical value measured in the comparison data and a numerical value measured in the target data, and determine the produced difference as a comparison value.

In operation S120, the electronic device 10 may identify an ROI in an object of interest included in the target data based on the comparison value and the target data. The electronic device 10 may select an object having a comparison value that is greater than or equal to a reference value among at least one object as an object of interest. The reference value may have a value that varies depending on the object.

For example, the electronic device 10 may identify types of a first object and a second object that are different from each other from the target data and the comparison data, compare a comparison value of the first object with a first reference value based on the type of the first object, and compare a comparison value of the second object with a second reference value based on the type of the second object.

The size or number of objects included in the medical data may have different values every day depending on the condition of the subject. In this case, an error range of different values may vary depending on the object characteristics, and the electronic device 10 according to the present disclosure may classify whether the state of the corresponding object is within the error range or in a dangerous state by setting different reference values for each object.

According to an embodiment, the electronic device 10 may input at least one object identified from the target data and the comparison data into a trained reference value determination model, and the reference value determination model may infer a reference value for each of the at least one object.

In operation S120, the electronic device 10 may identify an ROI in an object of interest included in the target data based on the comparison value and the target data. For example, when the electronic device 10 identifies that the comparison value of the first object is greater than or equal to the first reference value, the electronic device 10 may select the first object as an object of interest. In addition, when the electronic device 10 identifies that the comparison value of the second object is greater than or equal to the second reference value, the electronic device 10 may select the second object as an object of interest.

The electronic device 10 may display a portion of the object of interest of the target data obtained by subtracting the reference value from the comparison value as an ROI. When the target data is a medical report and the object of interest is an item of the medical report, a difference between a numerical value of the target data and a numerical value of the comparison data in the corresponding item may be produced, and then a numerical range obtained by subtracting the reference value from the difference may be displayed as an ROI. For example, when a target data value is 10 and a comparison data value is 5 in an item A of the medical report, and a reference value corresponding to the item A is 2, a numerical range from 7 to 10, which is a value obtained by subtracting the reference value of 2 from the difference between the target data and the comparison data of 5, may be displayed as an ROI.

When the target data is a medical image and the object of interest is a body tissue identified in the medical image, a difference between the size of the target data and the size of the comparison data in the body tissue may be produced, and then an image region obtained by subtracting the reference value from the difference may be set as an ROI. For example, when the size of the target data is 10 and the size of the comparison data is 5 in a body tissue A identified in the medical image, and a reference value corresponding to the body tissue A is 2, an image region corresponding to 3 obtained by subtracting the reference value 2 from the difference between the target data and the comparison data of 5 may be displayed as an ROI. Since the size of the target data is 10, the image region corresponding to 3 may be an image region from the edge of the object of interest to a point representing 30% of the object of interest.

In operation S130, the electronic device 10 may display the identified ROI in a specific display method. That is, the electronic device 10 may display the ROI in a different display method from the remaining region except the ROI. For example, the electronic device 10 may display the ROI through a graphic element having a specific RGB value in the target data displayed in grayscale.

According to an embodiment, the reference value may be set as a plurality of values, and the ROI may be displayed as a plurality of graphic elements based on the comparison between the comparison value and the plurality of reference values. Accordingly, the specialist or the subject may intuitively recognize the severity of the subject's condition from the target data.

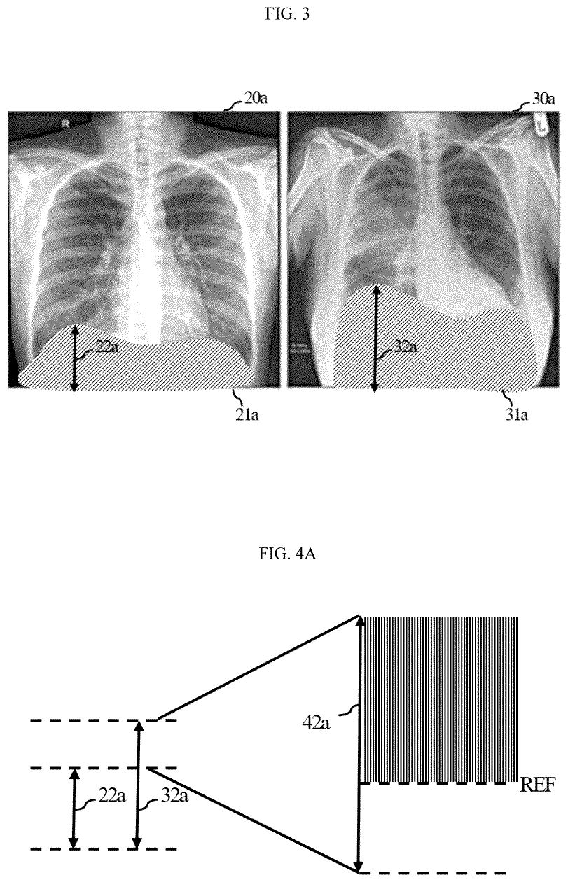

FIG. 3 is a diagram illustrating an embodiment in which an object of interest is extracted based on a comparison between images according to an embodiment.

Referring to FIG. 3, when comparison data 20a and target data 30a are medical images, the electronic device 10 may identify at least one object 21a and 31a from the medical images, and compare the sizes of the at least one object 21a and 31a.

The electronic device 10 may extract all types of identifiable objects from the medical image. For example, the electronic device 10 may extract the outlines of the objects, and extract whether the extracted outline corresponds to a certain object based on a trained object identification model. The object identification model may be a model trained based on training data labeled with outlines of body tissues and names of the body tissues corresponding to the outlines.

When the types of the object 21a extracted from the comparison data 20a and the object 31a extracted from the target data 30a are the same, the electronic device 10 may compare the sizes of the corresponding objects. The electronic device 10 may calculate the area of the object using a measuring tool, and may produce the difference between the calculate areas as a comparison value. Alternatively, the electronic device 10 may calculate the difference between an object length 22a in the comparison data and an object length 32a in the target data using a measuring tool.

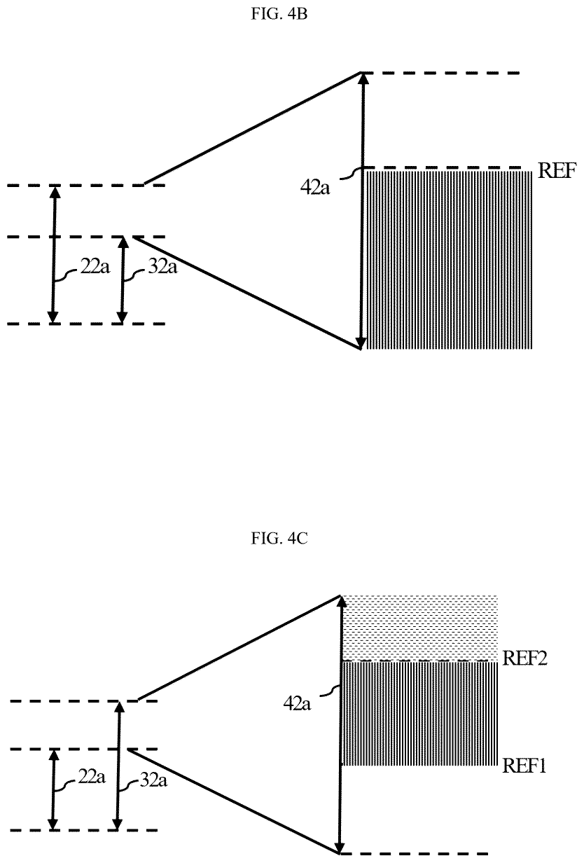

FIG. 4A to FIG. 4C are diagrams illustrating an embodiment in which an ROI is displayed based on a comparison between a comparison value and a reference value according to an embodiment.

Referring to FIGS. 4A and 4B, the electronic device 10 may compare a comparison value 42a, which is a difference between the object length 22a calculated in the comparison data and the object length 32a calculated in the target data, with a reference value REF. When the comparison value is less than or equal to the reference value REF, the electronic device 10 may not select the corresponding object as an object of interest. In other words, when it is identified that the difference between the object length 22a in the comparison data and the object length 32a in the target data is not large, the object may be excluded from being displayed as an ROI, thereby allowing unnecessary detailed review of the corresponding object to be omitted.

In contrast, when the comparison value is greater than the reference value REF, the electronic device 10 may select the corresponding object as an object of interest and display a portion of the selected object of interest as an ROI.

Referring to FIG. 4A, when the value of the target data is greater than the value of the comparison data, the electronic device 10 may display a portion from a region in the comparison value 42a that is greater than the reference value REF to the outline of the target data as an ROI. In other words, the electronic device 10 may display a portion from a region obtained by adding the reference value REF to the value of the comparison data to the outline of the target data as an ROI.

Referring to FIG. 4B, when the value of the comparison data is greater than the value of the target data, the electronic device 10 may display a portion from a region in the comparison value that is less than reference value REF to the outline of the target data as an ROI. In other words, the electronic device 10 may display a portion from a region obtained by subtracting the reference value REF from the value of the comparison data to the outline of the target data as an ROI.

Referring to FIG. 4C, the electronic device 10 may compare a comparison value with a plurality of reference values set for each object. The plurality of reference values may include a first reference value REF1 and a second reference value REF2 having a larger value than the first reference value REF1.

When the comparison value 42a is greater than or equal to the first reference value REF1, the electronic device 10 may display a region in an object of interest that is greater than or equal to the first reference value REF1 and less than the second reference value REF2 as a first ROI. When the comparison value is greater than or equal to the second reference value REF2, the electronic device 10 may display a region in an object of interest that is greater than or equal to the second reference value REF2 as a second ROI. In this case, the first ROI and the second ROI may be displayed in different display methods, and for example, the first ROI may be displayed in yellow, and the second ROI may be displayed in red.

According to an embodiment, when the comparison data is previous medical data for the target data, the electronic device 10 may adaptively set the reference value to intuitively display the degree of improvement in the condition of the subject. For example, the electronic device 10 may set a reference value based on a normal numerical range of an identified object and a numerical value of comparison data.

The electronic device 10 may compare a numerical value of the comparison data with the normal numerical range. The numerical value and the numerical range may be, but are not limited to, the size of an object identified in the medical image, and may be a blood concentration. When the electronic device 10 determines that the numerical value of the comparison data deviates from the normal numerical range to a degree greater than or equal to a reference ratio, the electronic device 10 may calculate a ratio of the numerical value of the comparison data with respect to one of the highest value and the lowest value of the normal numerical range. The electronic device 10 may set a new reference value by multiplying the previous reference value by the reciprocal of the ratio.

Referring to FIG. 4A, when the value of the target data is greater than the value of the comparison data and the numerical value of the comparison data is twice the highest value of the normal numerical range, the electronic device 10 may set a value obtained by multiplying the previous reference value by ½ as a new reference value. Referring to FIG. 4B, when the value of the target data is smaller than the value of the comparison data, and the numerical value of the comparison data is half the highest value of the normal numerical range, the electronic device 10 may set a value obtained by multiplying the previous reference value by 2 as a new reference value.

Accordingly, the electronic device 10 may, upon identifying that the comparison data, which is the previous medical data, is already outside the normal numerical range, apply a stricter reference value. The electronic device 10 may apply a stricter reference value and when improvement of the patient's condition is low, select the corresponding object as an object of interest, thereby adjusting the reference value to suit the situation.

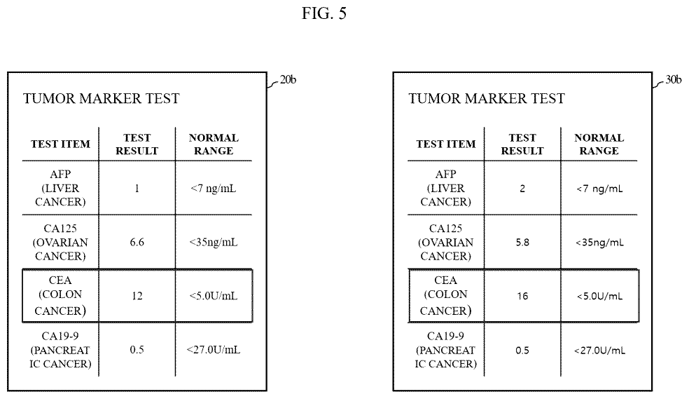

FIG. 5 is a diagram illustrating an embodiment in which an object of interest is extracted based on a comparison between medical reports according to an embodiment.

Referring to FIG. 5, an electronic device 10 according to an embodiment may, when comparison data 20b and target data 30b are medical reports, extract object information included in the medical reports, and may extract a numerical value corresponding to the extracted object information. For example, when the comparison data 20b and the target data 30b are reports on tumor marker tests, the electronic device 10 may extract object information for test items, that is, AFP (liver cancer), CA125 (ovarian cancer), CEA (colon cancer), and CA19-9 (pancreatic cancer). The electronic device 10 may extract test results corresponding to each of AFP (liver cancer), CA125 (ovarian cancer), CEA (colon cancer), and CA19-9 (pancreatic cancer) to match each item.

The electronic device 10 may produce the difference in test result values for each item between the comparison data 20b and the target data 30b as a comparison value. For example, when the value of the comparison data 20b corresponding to AFP (liver cancer) is 1 ng/mL and the value of the target data 30b corresponding to AFP (liver cancer) is 2 ng/mL, a comparison value corresponding to AFP (liver cancer) may be 1 ng/mL. Similarly, the electronic device 10 may produce a comparison value corresponding to CA125 (ovarian cancer) as −0.8 ng/mL, a comparison value corresponding to CEA (colon cancer) as 4 ng/mL, and a comparison value corresponding to CA19-9 (pancreatic cancer) as 0.

The electronic device 10 may compare the comparison value with a reference value set for each object, and may set an object of interest based on the comparison result to display an ROI. For example, the electronic device 10 may shade an item corresponding to an object of interest.

According to an embodiment, when an object of the comparison data 20b is a smaller object than an object of the target data 30b, the electronic device 10 may set a portion of the target data 30b corresponding to the object of the comparison data 20b as an object of interest. The electronic device 10 may not compare numerical values corresponding to objects, but instead classify a body tissue into a plurality of layers, and when the body tissue of the target data 30b corresponds to a higher level than the body tissue of the comparison data 20b, the electronic device 10 may identify the body tissue of the comparison data 20b in the target data 30b and then set the identified portion as an object of interest.

Meanwhile, the methods according to the various embodiments of the present invention described above may be implemented in the form of an application or software program that may be installed on an existing electronic device.

In addition, all or a part of the method may be configured as several software function modules and implemented in an OS. Alternatively, each operation may be configured as one software function module, or each operation may be combined to be configured as one software function module and implemented on an OS. Accordingly, even when some embodiments of the present disclosure are not implemented entirely as one software function module, when several software function modules implement each operation of the present disclosure and several software function modules are implemented in one OS, it should be understood that the method of the present disclosure has been implemented.

In addition, the methods according to the various embodiments of the present invention described above may be implemented only with a software upgrade or a hardware upgrade for an existing electronic device. In addition, the various embodiments of the present invention described above may also be performed through an embedded server with which the electronic device is equipped, or an external server of the electronic device.

Meanwhile, according to an embodiment of the present invention, the various embodiments described above may be implemented using software, hardware or a combination thereof, or as software including instructions stored in a computer readable recording medium that can be read by a computer or a similar device. In some cases, the embodiments described in this specification may be implemented by the processor itself. According to a software implementation, embodiments such as the procedures and functions described in this specification may be implemented as separate software modules. Each of the software modules may perform one or more functions and operations described in this specification.

Meanwhile, a computer or a similar device may be a device capable of invoking instructions stored in a storage medium and operating in accordance with the invoked instructions and may include a device according to the disclosed embodiments. When the instructions are executed by a processor, the processor may directly perform a function corresponding to the instructions or may use other components to perform the function under the control of the processor. The instructions may include code generated or executed by a compiler or an interpreter.

A recording medium that is read by a machine may be provided in the form of a non-transitory computer readable recording medium. Here, when a recording medium is referred to as “non-transitory,” it can be understood to mean that the recording medium is tangible and does not include a signal, not that data is semi-permanently or temporarily stored in the recording medium. A non-transitory computer readable medium is a medium that can store data semi-permanently and can be read by a device, rather than a medium that stores data for a short period of time, such as a register, cache, or memory. Examples of non-transitory computer readable media may include a compact disc (CD), a digital versatile disc (DVD), a hard disk, a Blu-ray disc, a Universal Serial Bus (USB), a memory card, and a read-only memory (ROM).

As is apparent from the above, the embodiments of the present disclosure can enable automatic setting of a region of interest (ROI) that needs to be carefully examined in target data by comparing the target data with comparison data. Accordingly, an analysis processor can be used for subsequent patient condition analysis using the automatically set ROI, and medical specialists can review medical data by focusing on a part set as the ROI without needing to examine all the medical data, thereby significantly reducing the analysis and review time.

The effects of the present disclosure are not limited to the effects described above, and other effects that are not described will be clearly derived and understood by those skilled in the art from the following description. That is, unintended effects resulting from implementing the exemplary embodiments of the present disclosure may also be derived from the exemplary embodiments of the present disclosure by a person having ordinary skill in the art.

The present invention has been described with reference to exemplary embodiments illustrated in the drawings and specification. Although the embodiments have been described using specific terms in the specification, the embodiments disclosed above should be construed as being illustrative rather than limiting the present invention, and those skilled in the art should appreciate that various substitutions, modifications, and changes are possible without departing from the scope and spirit of the present invention. Therefore, the scope of the present invention is defined by the appended claims of the present invention.

What is claimed is:

1. A method of displaying a region of interest (ROI) of target data based on a comparison of medical data, the method comprising:
producing a comparison value based on a difference between comparison data and the target data;
identifying an ROI in an object of interest included in the target data based on the comparison value and the target data; and
displaying the identified ROI in a different display method from remaining regions excluding the ROI.

2. The method of claim 1, wherein the identifying of the ROI includes:
selecting an object included in the target data as the object of interest when the comparison value is greater than or equal to a reference value that varies by object.

3. The method of claim 2, wherein the displaying includes:
comparing the comparison value with a plurality of reference values for the object;
when the comparison value is greater than or equal to a first reference value, displaying a region of the object of interest that is greater than or equal to the first reference value and less than a second reference value as a first ROI;
when the comparison value is greater than or equal to the second reference value which is larger than the first reference value, displaying a region of the object of interest that is greater than or equal to the second reference value as a second ROI; and
displaying the first ROI and the second ROI in different display methods.

4. The method of claim 2, wherein the selecting of the object of interest includes:
when the comparison data is a previous medical image for the target data, setting the reference value based on a normal numerical range for an object identified in the comparison data and the target data and a numerical value of the comparison data.

5. The method of claim 1, wherein the producing of the comparison value includes:
when the target data is a medical report, extracting object information included in the medical report;
extracting a numerical value corresponding to the object information from the medical report;
and
producing a difference between a numerical value included in the comparison data and the numerical value included in the target data as the comparison value.

6. The method of claim 1, wherein the producing of the comparison value includes:
when the target data is a medical image, identifying an object of the medical image;
measuring a size of the object using a measuring tool for each type of medical image; and
producing the comparison value based on the size of the measured object.

7. The method of claim 1, wherein the identifying of the ROI includes:
when an object of the comparison data is an object having a smaller size than an object of the target data, setting a portion of the target data corresponding to the object of the comparison data as an object of interest.
